(19) United States
(12) Patent Application Publication
(10) Pub. No.: US 2011/0124703 A1
(43) Pub. Date: May 26, 2011

(54) MOLECULES COMPRISING A BIS(HETEROARYL)MALEIMIDE BACKBONE, AND USE THEREOF IN THE INHIBITION OF DDE/DDD ENZYMES

(76) Inventors: Marie-Claude Viaud-Massuard, Tours (FR); Jérôme Guillard, Ligueil (FR); Yves Bigot, Saint Avertin (FR); Corinne Auge-Gouillou, Veretz (FR)

(21) Appl. No.: 12/735,667

(22) PCT Filed: Feb. 3, 2009
(86) PCT No.: PCT/EP2009/051210

§ 371 Date: Feb. 8, 2011

(30) Foreign Application Priority Data
Feb. 4, 2008 (FR) 08/00569

(51) Int. Cl. A61K 31/4025; C07D 405/14; C12N 9/99; C12N 7/06; C12Q 1/48; A61P 31/18

(52) U.S. Cl. 514/422; 548/517; 435/184; 435/238; 435/15

(57) ABSTRACT

The invention concerns molecules with a bis-(heteroaryl)maleimide structure and having inhibiting characteristics with respect to enzymes with a catalytic pocket comprising the invariant amino acids D, D and E or D, D and D, such as transposases, RAG recombinases or retroviral integrases. The invention also concerns the use of said molecules for in vitro, ex vivo or in vivo inhibition of transposases, RAG recombinases and retroviral integrases such as HIV integrase, as well as the use of said molecules in the treatment of diseases associated with these enzymes in an animal or human host, in particular in the treatment of AIDS.

### TECHNICAL FIELD

The invention relates to the field of enzyme inhibitors with a catalytic pocket comprising the invariant amino acids D, D and E or D, D and D, such as transposases or retroviral integrases, as well as to the use of these inhibitors in in vitro tests, or in the treatment of diseases associated with these enzymes in an animal or human host. The invention also relates to a system for screening said inhibitors, in particular in vitro, in eukaryotic cells.

### PRIOR ART

Transposases and retroviral integrases are enzymes that encourage the displacement of DNA segments within the same genome or between genomes. Such enzymes, although they derive from different organisms, have structurally similar catalytic domains. Thus, crystallographic studies have shown that despite the absence of similarity in their polypeptide sequences, the structure of the catalytic core (or pocket), in particular the positioning of a triad of invariant DDE/D residues, is broadly superimposable for at least three transposases (MuA, Tn5 and MOS1) (10, 12) and at least two integrases (HIV-1 and ASV) (12, 13). In all of those enzymes, the role of the catalytic triad is to facilitate the catalysis by coordinating the metal ions necessary therefore. It has also recently been demonstrated that Tn5 transposase can be used to identify HIV-1 integrase inhibitors (14, 15).

DNA transposons are class II transposable elements that correspond to discrete DNA segments which are “naturally” susceptible of being moved within genomes (for an overview, see (1)). Further, experience has shown that certain of them have a large capacity for being moved in species that are only slightly related to the host in which they were initially isolated. Those properties have led to the development of tools based on transposons for insertional mutagenesis and germinal transgenesis in model organisms, and potentially for gene therapy. The transposons Mos1 (2), Himar1 (3), Minos (4), PiggyBac (5), Sleeping Beauty (6) and Tol2 (7) have been selected as principal candidates for the development of such tools (transposon tools).

Mos1 is a 1286 bp element terminated by 28 bp inverted terminal repeats (ITR). Mos1 contains a single open reading frame coding for MOS1 (a 345 amino acid transposase) and moves along the genome of its hosts by means of a cut and paste mechanism. The mechanism in its entirety is composed of four principal steps: [1 and 2] homodimerisation of MOS1 and assembly of a synaptic complex, [3] excision of Mos1, and [4] target recognition and insertion of Mos1 in a new locus (FIG. 2). This activity is based on a triad of amino acids, DD34D, which chelate the cations necessary for catalysis (10). Mos1 transposase belongs to the large “DDE enzymes” family (11). In that group, mariner transposases constitute an exception as they comprise a DDD triad.

Understanding the activity of transposases and monitoring the efficacy of transposition in the context of “transposon tools” involves identifying molecules capable of inhibiting the activity of those enzymes. However, until now, none of those compounds has been available for mariner transposases.

Retroviral integrase (coded by the pol gene of a retrovirus) is an enzyme that ensures integration of the viral genome (retrotranscribed in the form of a double strand DNA) into the genome of the infected cell (target DNA). Integrase, in fact, carries out two enzymatic functions in the integration process: cleavage of DNA and strand transfer. Thus, integrase (IN) recognizes and binds the viral site att located at the end of the retroviral LTRs (long terminal repeat), and catalyzes the excision of two base pairs in the 3′ portion of those LTRs. Once cleaved, the viral DNA is imported, via a protein/DNA complex (PIC or pre-integration complex), into the nucleus of the host cell where the integrase then cleaves the target DNA to form free 5′ ends which are bound to the free 3′ OH portions of the viral DNA. No specificity vis-à-vis the nucleotide sequence of the target DNA, where cleavage and integration of viral DNA has occurred, has been identified.

The catalytic domain of retroviral integrases contains the invariant triad with motif DD-35-E, respectively comprising the D64, D116 and E152 residues in the case of HIV-1 integrase or D64, D121 and E157 residues in the case of Roux sarcoma virus (RSV).

Because of their role in retroviral diseases, understanding the mechanisms linked to the activity of retroviral integrases leading to the integration of retroviruses into infected cells, whether they be animal or human in origin, as well as the identification of molecules capable of inhibition in vivo, for therapeutic purposes, is a public health priority.

Thus, there is a real need for the identification of molecules that are capable of inhibiting transposases, but also retroviral integrases. These molecules can be used both in comprehending the mechanisms of the excision and integration steps and in the treatment of symptoms associated with infection in animals or patients by retroviruses. Investigation of said molecules also necessitates characterization of a DDE/DDD enzyme inhibitor screening system capable of functioning both in a prokaryotic and in a eukaryotic environment in order to ensure that the molecules that are identified thereby have inhibiting activities that are susceptible of being explored with a view to applications in therapy.

### DETAILED DESCRIPTION OF THE INVENTION

The present invention envisages molecules which have a common bis-(heteroaryl)maleimide structure and have the following formula I:

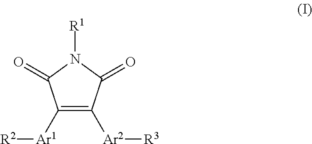

In formula (I), the group R¹ is selected from the group containing a hydrogen, a linear or branched alkyl, an alkenyl, an alkynyl, a cycloalkyl, a heterocyclyl, a heteroaryl, a heteroaralkyl, a heteroalkoxy, a carboxyl, an alkoxycarbonyl, a tetrazolyl, an acyl, an arylsulphonyl, a heteroarylsulphonyl, a phenyl, a hydroxyphenyl or a group:

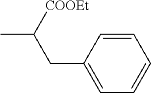

In a particular embodiment, R′ is H.

In another embodiment, R¹ is a phenyl group, optionally substituted, such as 4-hydroxyphenyl.

In another embodiment, R¹ has the following formula:

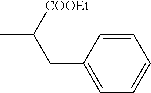

In said formula (I), the group R² and the group R³ are selected, independently of each other, from the group constituted by a hydrogen atom, a carboxylic acid (preferably C1 to C5), a cyano, an oxime, an oxime ether, a tetrazole, an ester, a substituted or unsubstituted amide, an acid (preferably C1 to C5), a dibasic acid (preferably C1 to C5), a cycloalkylcarboxylic acid (in particular C5 or C6), a substituted or unsubstituted aryl, a substituted or unsubstituted heteroaryl and the group —C═CH—CHO.

In a particular embodiment, R² or R³ is COOH.

In another embodiment, R² or R³ is C═CH—COH.

In this formula (I), Ar¹ and Ar² are selected, independently of each other, and are constituted by a substituted or unsubstituted cycloalkyl (C5 or C6), a substituted or unsubstituted heteroaryl, a substituted or unsubstituted aryl, and a linear or branched heteroalkyl. The cycloalkyl may also comprise one or more heteroatoms selected from N, O and S.

In a particular embodiment, Ar¹ and/or Ar² are selected, independently of each other, from substituted or unsubstituted monocyclic heteroaryls. In particular, Ar¹ is constituted by a monocyclic heteroaryl carrying R², selected from the group R²-pyridyl, R²-pyrazinyl, R²-furanyl, R²-thienyl, R²-pyrimidinyl, R²-isoxazolyl, R²-isothiazolyl, R²-oxazolyl, R²-thiazolyl, R²-pyrazolyl, R²-furazanyl, R²-pyrrolyl, R²-pyrazolyl, R²-triazolyl, R²-pyrazinyl and R²-pyridazinyl, said monocyclic heteroaryl being substituted or unsubstituted. In addition, In a particular embodiment, Ar², independently of Ar¹, is constituted by a monocyclic heteroaryl carrying R³, selected from the group R³-pyridyl, R³-pyrazinyl, R³-furanyl, R³-thienyl, R³-pyrimidinyl, R³-isoxazolyl, R³-isothiazolyl, R³-oxazolyl, R³-thiazolyl, R³-pyrazolyl, R³-furazanyl, R³-pyrrolyl, R³-pyrazolyl, R³-triazolyl, R³-pyrazinyl and R³-pyhdazinyl, said monocyclic heteroaryl being substituted or unsubstituted.

In a particular embodiment, Ar¹ or Ar² is a furan. In another embodiment, Ar¹ and/or Ar² are respectively a R²-furanyl group and a R³-furanyl group. In particular, Ar¹ and/or Ar2, independently of each other, are respectively R²-fur-2-yl, R²-fur-3-yl or R²-fur-4-yl and R³-fur-2-yl, R³-fur-3-yl or R³-fur-4-yl. In a preferred mode, Ar¹ and Ar² are respectively R²-fur-2-yl and R³-fur-2-yl.

In a particular embodiment of the invention, where the groups Ar¹ and Ar² are furan, in particular fur-2-yl, the groups R² and/or R³, in particular R² and R³, are carried in the 3, 4 or 5 position, in particular in the 5 position.

In particular, the application envisages a molecule comprising a bis-(heteroaryl)maleimide structure with formula (I):

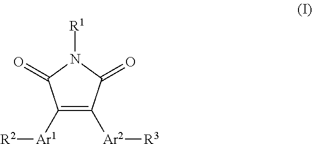

in which:
R¹ is selected from the group constituted by a hydrogen, a linear or branched alkyl, an alkenyl, an alkynyl, a cycloalkyl, a heterocyclyl, a heteroaryl, a heteroaralkyl, a heteroalkoxy, a carboxyl, a alkoxycarbonyl, a tetrazolyl, an acyl, an arylsulphonyl, a heteroarylsulphonyl, a phenyl, a hydroxyphenyl or a group:

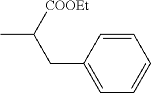

R² and the R³ are selected, independently of each other, from the group constituted by a hydrogen atom, a carboxylic acid, a cyano, an oxime, an oxime ether, a tetrazole, an ester, a substituted or unsubstituted amide, an acid, a dibasic acid, a cycloalkylcarboxylic acid, a substituted or unsubstituted aryl, a substituted or unsubstituted heteroaryl and the group —C═CH—CHO;
Ar¹ and Ar² are selected independently of each other and are constituted by a substituted or unsubstituted cycloalkyl, a substituted or unsubstituted heteroaryl, a substituted or unsubstituted aryl, or a linear or branched heteroalkyl; and in which it is excluded that R¹, R² and R³ are together H.

In the context of the present invention, it is explicitly excluded that the groups R¹, R² and R³ are together a hydrogen atom (H) in the compound with formula (I).

In a particular embodiment, the following compounds are also explicitly excluded from the definition given for the molecules of the invention:
a molecule with formula (I) in which when R¹ is a hydrogen and Ar¹ and Ar² are furans, R² and R³ are CHO; and
a molecule with formula (I) in which when R¹ is a phenyl and Ar¹ and Ar² are furans, R² and R³ are CHO.

In a particular embodiment, R² and R³ are identical, and the molecule of the invention has the following formula (Ia):

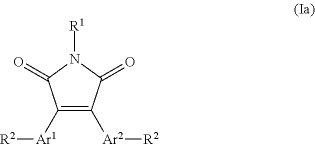

in which R¹, R², Ar¹ and Ar² are as defined above.

In a particular embodiment, Ar¹ and Ar² are identical and the molecule of the invention has the following formula (Ib):

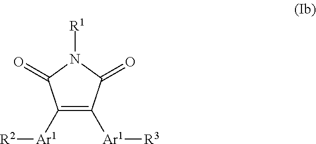

in which R¹, R², R³ and Ar¹ are as defined above.

In another embodiment of the invention, R² and R³ on the one hand, and Ar¹ and Ar² on the other hand are identical, and the molecule of the invention has the following formula (Ic):

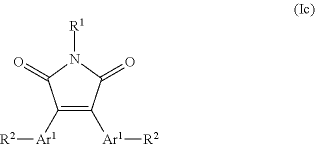

in which R¹, R² and Ar¹ are as defined above.

A particular molecule of the invention is a molecule in which Ar¹ and Ar² are a furan, and which has the following formula (II):

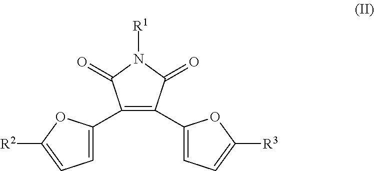

in which R¹, R² and R³ are as defined above.

In a particular embodiment, the molecule with formula (II) has identical groups R² and R³ and has the following formula (IIa):

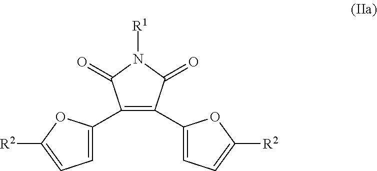

in which R¹ and R² are as defined above.

The particular embodiments defined in the present application, in particular in relation to formula (I), also apply in the same manner to formulae (Ia), (Ib), (Ic), (II) and (IIa).

In a particular embodiment, the molecule of the invention in accordance with formula (I), (Ia), (Ib), (Ic), (II) or (IIa) is “N-substituted”, i.e. the group R¹, in the definition given above, is different from a hydrogen atom. In this case, the nature of groups R² and R³ is selected from the possibilities given above.

In another embodiment, an alternative to the foregoing, the molecule of the invention in accordance with formula (I) (Ia), (Ib), (Ic), (II) or (IIa) is not N-substituted (and is thus qualified as a “non-N-substituted” molecule), i.e. the group R¹ is a hydrogen atom. In this case, the groups R² and R³, independently of each other, satisfy one of the possibilities given above, with the exception of the hydrogen atom (since R¹, R² and R³ cannot together be a hydrogen atom).

An example of the synthesis of particular molecules of the invention is shown in FIG. 11. Thus, the application also envisages any molecule having formula (I) and obtained by a similar or identical synthesis.

The invention also envisages molecules described in the present application in the form of physiologically acceptable salts. Examples of physiologically acceptable salts include basic salts such as salts of alkali metals (for example sodium or potassium) or salts of alkaline-earth metals (for example calcium), basic organic salts and amino acid salts. The embodiments defined or illustrated with reference to molecules (I), (Ia), (Ib), (Ic), (II) or (IIa) are also applicable to physiologically acceptable salts of said molecules.

In the context of the present invention, the following definitions apply to the description and to the claims.

The term “alkyl” means a hydrocarbon chain, linear or branched, containing 1 to 20 carbon atoms, preferably 1 to 12 carbon atoms, more preferably 1 to 6 carbon atoms.

The term “alkenyl” means a linear or branched aliphatic hydrocarbon group containing at least one carbon-carbon double bond and 2 to 20 carbon atoms, preferably 2 to 12 carbon atoms, more preferably 2 to 6 carbon atoms. Non-limiting examples of alkenyl groups are: ethenyl, propenyl, n-butenyl, n-pentenyl, octenyl and decenyl.

The term “alkynyl” means a linear or branched aliphatic hydrocarbon group containing at least one carbon-carbon triple bond, and 2 to 15 carbon atoms, preferably 2 to 12 carbon atoms, more preferably 2 to 4 carbon atoms. Non-limiting examples of alkynyl groups are: ethynyl, propynyl, 2-butynyl, 3-methylbutynyl, n-pentynyl and decynyl.

The term “aryl” means a monocyclic or multicyclic aromatic group system comprising at least one aromatic ring containing 6 to 14 carbon atoms, and preferably 6 to 10 carbon atoms. A non-limiting example of an aryl group is the phenyl group. The aryl group may be unsubstituted or substituted with 1, 2 or 3 substituents selected independently of each other. A non-limiting example of a substituted aryl group is hydroxyphenyl.

The term “arylalkyl” means an aryl group as defined above bonded to an alkyl group as defined above, the bond to the parent group being formed via the alkyl group. A non-limiting example of an arylalkyl group is benzyl.

The term “cycloalkyl” means a saturated carbocyclic system containing 3 to 10 (for example 3 to 7) carbon atoms, preferably 5 to 10 carbon atoms, and more preferably 5 to 7 carbon atoms and containing 1, 2 or 3 cycles. Non-limiting examples of a cycloalkyl group are cyclopropyl, cyclopentyl, cyclohexyl and cycloheptyl.

The term “halogen” means the groups fluorine, chlorine, bromine or iodine.

The term “heteroaryl” means a system of 5 to 14, preferably 5 to 10, simple or fused aromatic rings, said rings comprising 1, 2 or 3 heteroatoms independently selected from O, S and N, with the rings not having adjacent oxygen atoms or sulphur atoms. A preferred heteroaryl group contains 5 or 6 atoms. In another preferred embodiment, the heteroaryl is monocyclic. The heteroaryl group may be unsubstituted or substituted with 1, 2 or 3 substituents selected independently of each other. Non-limiting examples of heteroaryl groups are: pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, and benzothiazolyl. Preferred examples of monocyclic heteroaryls are pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, pyrazinyl and pyridazinyl. In particular, in the context of the invention, the preferred monocyclic heteroaryl is furanyl. The monocyclic heteroaryl may be unsubstituted or substituted with 1, 2 or 3 substituents selected independently of each other.

The term “heterocyclyl” or “heterocycloalkyl” means a saturated, monocyclic or multicyclic non-aromatic system comprising 3 to 10 (for example 3 to 7) carbon atoms, preferably 5 to 10 carbon atoms, and more preferably 5 to 7 carbon atoms and containing 1, 2 or 3 cycles, in which one or more of the atoms of the system is an element other than a carbon atom, such as a sulphur, oxygen and/or nitrogen atom.

The term “arylsulphonyl” means a-SO₂-aryl group, in which the aryl group is as defined above.

The term “heteroaralkyl” or “heteroarylalkyl” or respectively “heteroarylsulphonyl” means a heteroaryl group as defined above bonded to an alkyl group as defined above, respectively to a sulphonyl group as defined above, the bond to the parent group being made via the alkyl or respectively the sulphonyl group.

The term “alkoxy” means a —O-alkyl group, in which the alkyl group is as defined above, containing 1 to 20 carbon atoms, more preferably 1 to 10 carbon atoms.

The term “carboxyl” means a —C(O)OH functional group.

The term “acyl” means a —C(O)-alkyl group, in which the alkyl group is as defined above.

The term “substituted” or “substituent” means a halogen group, an ether, a hydroxyl, a carboxylic function, a carboxamide, an ester, a ketone, an aryl, a heteroaryl, a cycloalkyl, an amine, a substituted amine, a linear or branched alkyl, in particular a C1 to C6 alkyl, a cyano, a nitro, a haloalkyl, an alkoxy, a carboxyalkyl, a mercapto, a sulphhydryl, an alkylamino, a dialkylamino, a sulphonyl or a sulphonamido. In a particular embodiment, when a heteroaryl is substituted, in particular a monocyclic heteroaryl, the substitution takes place on the heteroatom(s) of the cycle. Alternatively or in combination with the preceding embodiment, when a heteroaryl is substituted, in particular a monocyclic heteroaryl, the substitution takes place on the carbon atom or atoms of the cycle.

The molecules of the invention having the definition given above have inhibiting properties for at least one DDE/DDD enzyme, in particular measured by an inhibiting activity observed in vitro. The experimental conditions which have been able to verify the inhibiting activity of the molecules of the invention on DDE/DDD enzymes, in particular on transposases and/or retroviral integrases, are given in the examples below.

The expression “DDE/DDD enzyme” as used in the context of the present application means any enzyme with a phosphatidyltransferase activity and which has a catalytic enzymatic site formed by the three amino acids D, D and E (DDE enzyme) or D, D and D (DDD enzyme). In a particular embodiment, the DDE/DDD enzymes are enzymes which encourage the movement of a DNA segment in a genome or between genomes. This expression encompasses transposases (bacterial or eukaryotic) and/or retroviral integrases and/or RAG recombinases.

In a particular embodiment, the DDE/DDD enzyme is a transposase of prokaryotic origin, in particular of bacterial origin, such as MuA, Tn5 or Tn10. In another embodiment, the DDE/DDD enzyme is a transposase of eukaryotic origin such as a transposase from the Tc1/mariner family (an example of which is the MOS1 transposase or the Sleeping Beauty transposase) or a transposase from the hAT family (an example of which is the Hermes transposase).

In another embodiment of the invention, the DDE/DDD enzyme is an integrase of retroviral origin, in particular an integrase coded by the pol gene of a retrovirus. Retroviruses which may be targeted by inhibiting molecules for the integrase of the invention belong to the genus Alpharetrovirus, Betaretrovirus, Gammaretrovirus, Deltaretrovirus, Lentivirus, and Spumavirus.

Particularly interesting lentiviruses are listed in Table I below:

TABLE I
list of lentiviruses

| | |
|---|---|
| Lentivirus | Human immunodeficiency virus 1 [HIV-1] |
| | Human immunodeficiency virus 2 [HIV-2] |
| | Simian immunodeficiency virus [SIV] |
| | Bovine immunodeficiency virus [BIV] |
| | Equine infectious anaemia virus [EIAV] |
| | Feline immunodeficiency virus |
| | Caprine arthritis encephalitis virus [CAEV] |
| | Visna/maedi virus [VISNA] |

In a particular embodiment, the retroviral integrase targeted by the molecules of the invention originates from a retrovirus with animal or human tropism.

In a particular embodiment, the retroviral integrase targeted by the molecules of the invention is from a lentivirus with human tropism, such as the HIV virus (human immunodeficiency virus), in particular the HIV-1 isolate and/or HIV-2 isolate (cytopathogenic retroviruses).

Particular molecules that inhibit the enzymatic activity of DDE/DDD enzymes, in particular transposases or retroviral integrases, are molecules with one of formulae (III) to (VI) below:

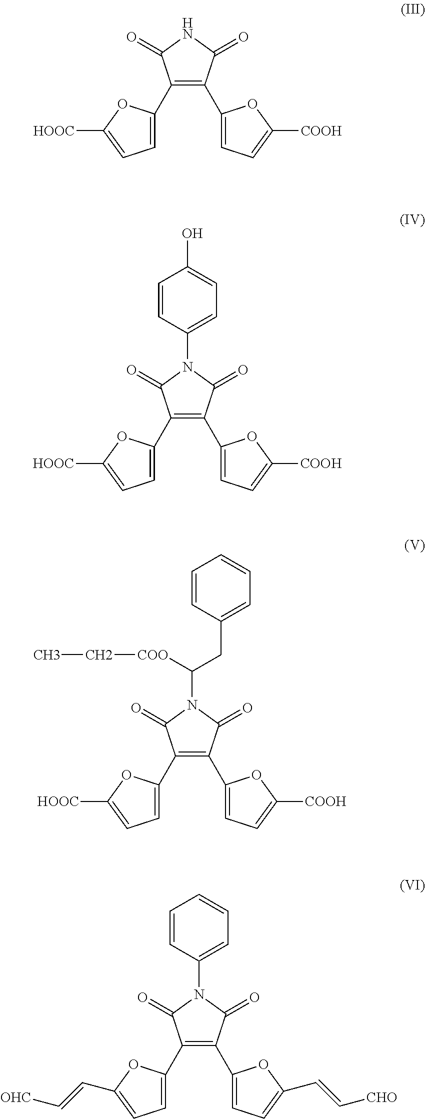

The invention also envisages a composition comprising at least one molecule as defined in the context of the present application, and in particular at least one molecule with formula (I), (Ia), (Ib), (Ic), (II), (IIa) above, or with one of formulae (III) to (VI) above.

In a particular embodiment of the invention, the composition comprises at least 2 different molecules selected from formulae (I) to (VI).

In a particular embodiment, the composition is a therapeutic or pharmaceutical composition, i.e. it is adapted for administration to an animal or to a patient.

In a particular embodiment, the composition of the invention also comprises a pharmaceutically acceptable vehicle. The term “vehicle” means any substance that allows the molecules of the invention to be formulated in a composition. In a particular embodiment, the vehicle is a substance or a combination of pharmaceutically acceptable substance(s), i.e. appropriate for use of the composition in contact with a living being (for example an animal, in particular a non-human mammal, and for example a human being), and is thus preferably non toxic. Examples of such pharmaceutically acceptable vehicles are water, a saline solution, solvents which are miscible with water, sugars, binders, excipients, pigments, vegetable or mineral oils, water-soluble polymers, surfactants, thickening agents or gelling agents, cosmetic agents, preservatives, alkalinizing or acidifying agents, etc.

In a particular embodiment, in addition to comprising at least one molecule of the invention and optionally one or more pharmaceutically acceptable vehicle(s), the composition of the invention further comprises an additional biologically active compound for the treatment of symptoms linked to infection with a retrovirus, in particular an additional compound that is biologically active in the treatment of symptoms linked to acquired immunodeficiency syndrome (AIDS). The term “additional” in the context of the present composition indicates that the compound is different from the molecules defined in the present application, in particular different from a molecule satisfying formula (I) above.

Examples of additional biologically active compounds for the treatment of symptoms linked to acquired immunodeficiency syndrome which may be cited are (a) protease inhibitors, (b) nucleosidic reverse transcriptase inhibitors, (c) non-nucleosidic reverse transcriptase inhibitors, (d) nucleotidic reverse transcriptase inhibitors, (e) nucleotidic and nucleosidic inhibitors, (f) integrase inhibitors, (g) fusion inhibitors and (h) interleukins.

The invention also concerns the use of at least one molecule described in the present application for the in vitro inhibition of the activity of DDE/DDD enzymes, in particular transposases or retroviral integrases. The term “in vitro” means an inhibition test carried out outside the entire living organism, i.e. both a test carried out in a test tube and a test carried out on a culture of prokaryotic or eukaryotic cells.

The invention also concerns a molecule or a composition in accordance with the invention for use as a drug.

Thus, it is possible to envisage the use of molecules or compositions of the invention for inhibiting the enzymatic activity of DDE/DDD enzymes, in particular the activity of transposases or retroviral integrases in accordance with the definition given in the present application. For this reason, the invention also envisages a molecule or a composition described in the present application, for use as DDE/DDD enzyme inhibitors. In this therapeutic context, the molecules, alone or used in the form of a composition, are N-substituted or non-N-substituted.

In a particular embodiment, the DDE/DDD enzymes are transposases or RAG recombinases. In a particular embodiment, the molecules or compositions of the invention are used for the ex vivo inhibition of the activity of the transposases or RAG recombinases.

In another embodiment, the DDE/DDD enzymes are integrases deriving from a retrovirus, in particular lentivirus, with animal or human tropism. Due to their involvement in the propagation of acquired immunodeficiency syndrome (AIDS), HIV type retroviruses such as those of types HIV-1 and/or HIV-2 (regardless of their sub-type), and the integrases of said retroviruses are particularly interesting targets for applications of the molecules of the invention

The invention also pertains to a molecule or to a composition of the invention for use in the treatment of diseases or symptoms associated with and/or consecutive upon an infection by a retrovirus, in particular consecutive upon an infection by a retrovirus with animal tropism or human tropism such as HIV.

In the context of the present application, the term “treatment” means both the curative effect (disappearance of the retrovirus, for example) obtained with at least one molecule or a composition of the invention, and an improvement in symptoms observed in the animal or patient (and consecutive upon or linked to the presence of a retrovirus) or an improvement in the condition of the patient. Thus, the term “treatment” is applicable to infection by the retrovirus as well as to symptoms or diseases resulting from infection by that retrovirus. Thus, a method comprising administration of a compound or a composition of the invention to an animal or a patient in need thereof for the treatment of diseases or symptoms associated with or consecutive upon an infection by a retrovirus also forms part of the invention.

The term “animal” in particular means a non-human mammal.

In a particular embodiment, the application concerns a molecule or composition of the invention for use in reducing or even suppressing retroviral replication. The effect of the molecule or the composition of the invention on the reduction or even suppression of replication of the retrovirus may be demonstrated by the change in the viral load in the plasma in the infected host

In another embodiment, the application envisages a molecule of the invention or a composition comprising it in the treatment of symptoms and/or infection consecutive upon an infection by HIV, and in particular in the treatment of acquired immunodeficiency syndrome (AIDS). Advantageously, the treatment discussed in this application is appropriate for HIV-1 and/or HIV-2 isolates. In the case of treatment of an infection by the isolate HIV-1, the treatment is applicable to isolates from group 0 and also to isolates from group M, in particular for isolates from the group M in clades A, B, C, D, E, F, G and H. The efficacy of treatment of diseases or symptoms linked to AIDS by the molecule or the composition of the invention may be determined by the change in the number of T CD4⁺ lymphocytes, the principal target cells of the HIV virus, optionally in combination with the computation of the change in viral load in the plasma.

In the therapeutic uses or therapeutic methods described above, the molecule or the composition of the invention may be in the solid form (cachet, powder, gelule, pill, suppository, quick release tablet, gastro-resistant tablet, delayed release tablet) or liquid (syrup, injectable solution, eye wash). Thus, depending on its galenical form, the molecule or the composition of the invention may be administered orally, buccal transmucosally, vaginally, rectally, parenterally (intravenously, intramuscularly or subcutaneously), transcutaneously (or transdermally or percutaneously) or cutaneously.

The use of a molecule or a composition of the invention in the manufacture of a drug for the treatment of symptoms and/or of the infection consecutive upon an infection by a retrovirus, in particular HIV, and in particular for the treatment of acquired immunodeficiency syndrome, also forms part of the invention.

In another aspect, the invention also pertains to the use of the MOS1 system (Mos1 transposon) as a DNA transfer tool (“transposon tool”), in particular in gene therapy.

In the context of the present application, the MOS1 system comprises the following elements:
(a) the MOS1 protein (amino acids 1 to 345), in particular fused to the maltose binding protein or MBP (to thereby increase stability but without modifying the activity) or an advantageous mutant of the MOS1 protein;
(b) the transposon, optionally with a portion of its sequence deleted and/or carrying a transgene of interest.

In a particular embodiment, the transfer of DNA is carried out in eukaryotic cells; the cells from that transfer could be used for ex vivo applications, such as in gene therapy, or to obtain factory cells (cells producing a protein of economic or medical interest).

The molecules of the invention may thus be used to stop the transfer reaction caused by bringing a MOS1 system as described above into contact with the cells to be transformed. The advantage of the molecules of the invention is thus the ability to control and avoid “cascade” reactions of the MOS1 system.

### DESCRIPTION OF THE DRAWINGS

FIG. 1: Structure of selected compounds 1 to 10.

FIG. 2: Model of the Mos1 transposition mechanism.

FIG. 3: Excision test on compound 9:
(a) In vitro excision tests were carried out using supercoiled (SC) pBC-3T3 and various concentrations of compound 9 as indicated in the top of the figure. (−): absence of transposase; (D): DMSO. The molecular weight markers for DNA (MW) are indicated in the left hand margin in kbp. The various products are indicated on the right hand side: OC (open circle), L (linear=4.6 kb), SC (supercoiled). The excised transposon (3T3=1.2 kb) and the plasmid backbone (pBC=3.4 kb) are also indicated.
(b) IC50 value plot; the percentage inhibition of the excision (ordinate) was determined as a function of the concentration (M) of compound 9 (abscissa).

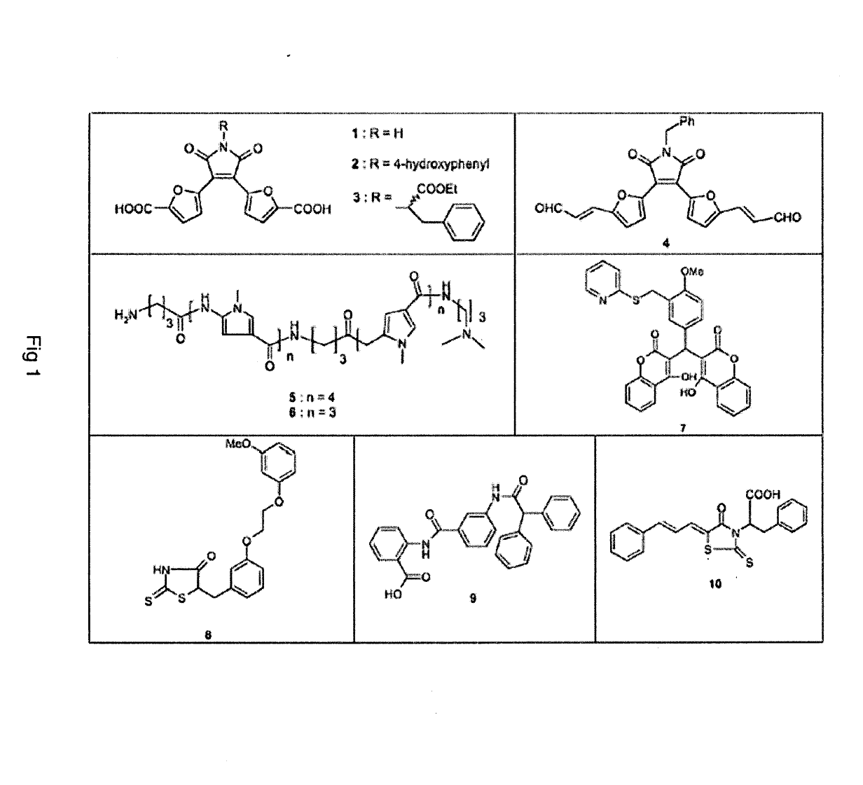

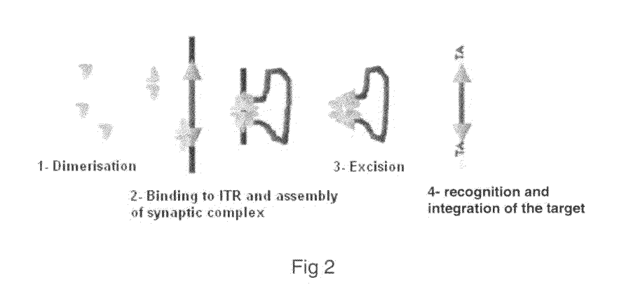

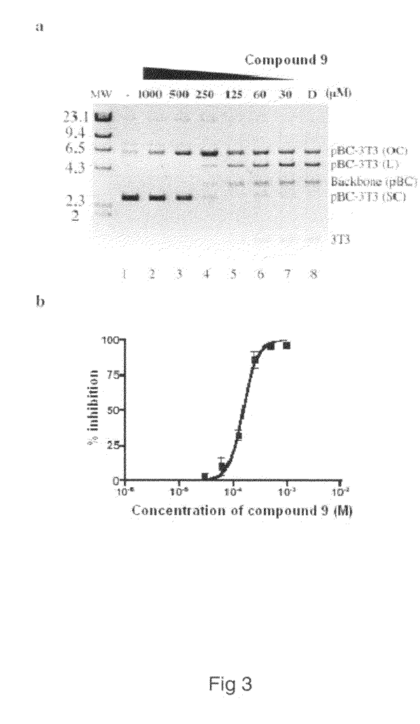

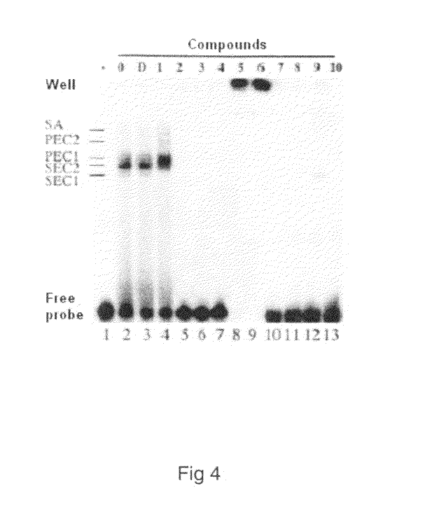

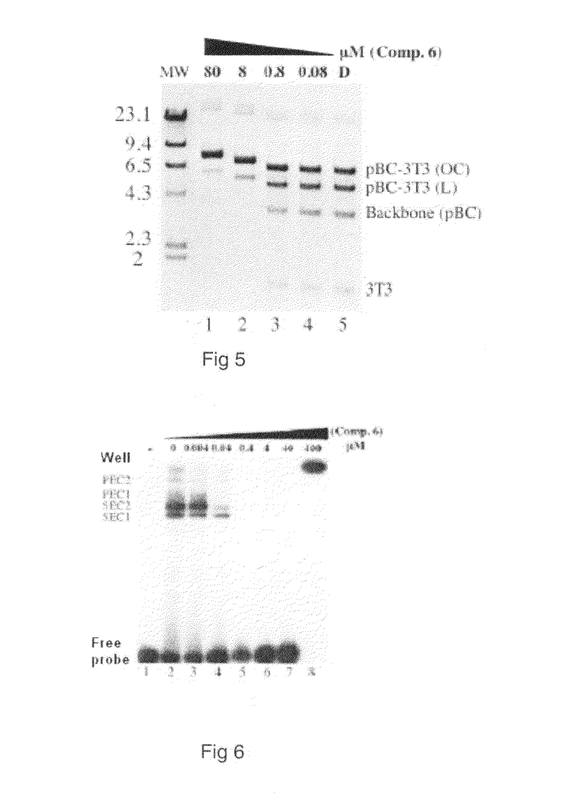

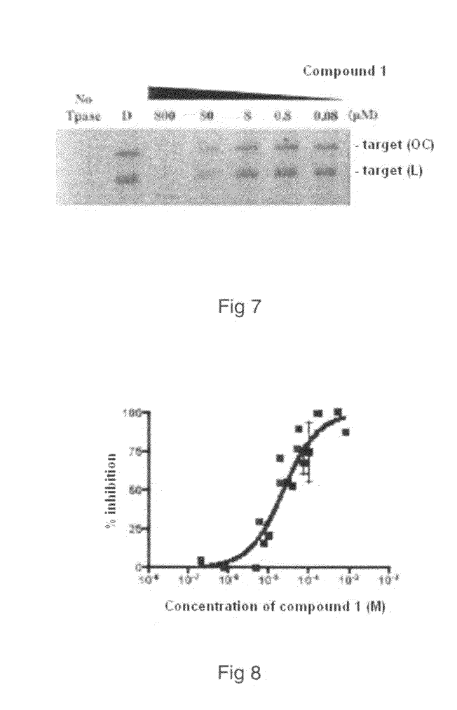

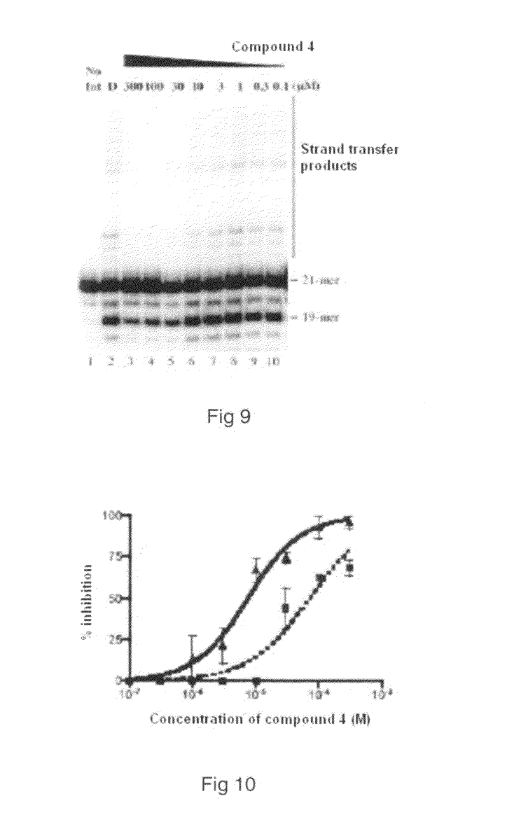

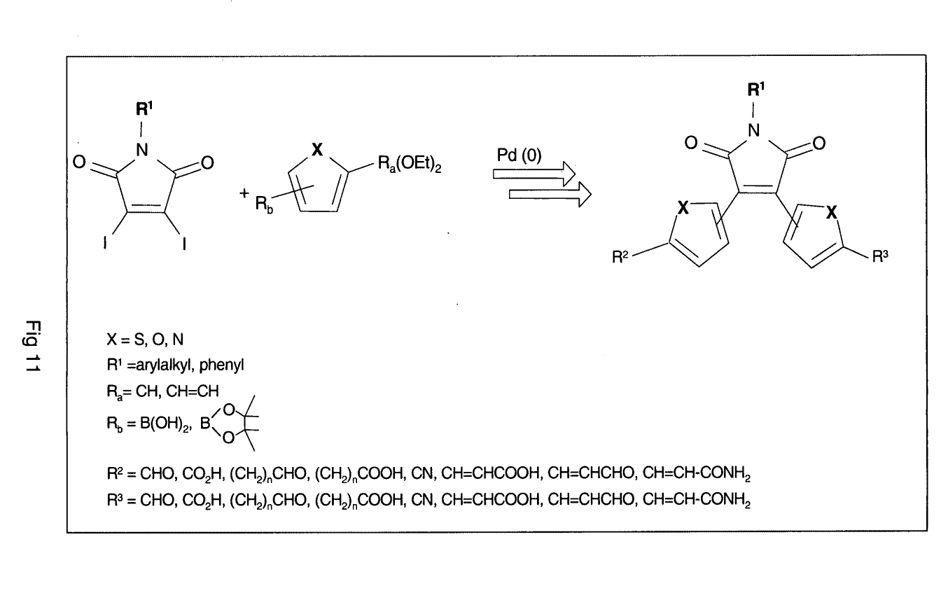

FIG. 4: Assembly of transposase-ITR complexes by EMSA (Electrophoretic-mobility shift assay). An EMSA was carried out with MBP-MOS1, using ITR30 as the probe and the selected 10 compounds (1 to 10 of FIG. 1). (−): absence of transposase; (0) no compound; (D): DMSO The various complexes (SEC1, SEC2, PEC1, PEC2), the target capture complex (TCC) and unbound DNA (free probe) are indicated. SEC1 was formed by a single transposase bound to a single ITR. This complex changed to result from the dissociation of the upper molecular complexes ((18) and personal communications). SEC2 was formed by a transposase dimer bound to a single ITR, PEC1 was formed by a transposase dimer bound to an ITR pair, and PEC2 was formed by a transposase tetramer bound to an ITR pair.

FIG. 5: Test for excision with compound 6. In vitro excision tests were carried out using supercoiled (SC) pBC-3T3 and various concentrations of compound 6 as indicated in the top of the figure: (D): DMSO. The DNA molecular weight markers are indicated in the left hand margin in kbp. The various products are indicated on the right hand side: OC (open circle), L (linear=4.6 kb), SC (supercoiled). The excised transposon (313=1.2 kb) and the plasmid backbone (pBC=3.4 kb) are also indicated.

FIG. 6: Effect of compound 6 on the assembly of MOS1-ITR complexes. An EMSA was carried out with MBP-MOS1, using NTR30 as the probe and various concentrations of compound 6 as indicated at the top of the figure. (−): absence of transposase; (0) no compound. The various complexes (SEC1, SEC2, PEC1, PEC2) and unbound DNA (free probe) are indicated. SEC1 was formed by a single transposase bound to a single ITR. That complex changed to result from dissociation of the higher molecular complexes. SEC2 was formed by a transposase dimer bound to a single ITR, PEC1 was formed by a transposase dimer bound to an ITR pair, and PEC2 was formed by a transposase tetramer bound to an ITR pair.

FIG. 7: MOS1 strand transfer reactions in the presence of compound 1. Tests were carried out using MBP-MOS1, labelled ITR30, pBC-SK as the target plasmid and various concentrations of compound 1 (in μM) as indicated at the top of the figure. No Tpase: absence of transposase; (D): DMSO. The integration products are indicated on the right: OC (open circle), L (linear).

FIG. 8: MOS1 strand transfer reactions. IC50 value plot; the percentage inhibition of excision (ordinates) was determined as a function of the concentration (in M) of compound 1 (abscissa). The IC50 value obtained is shown in Table III.

FIG. 9: Activities for 3′ maturation and for strand transfer for HIV-1 in the presence of compound 4; the tests were carried out using the HIV-1 integrase, an oligonucleotide duplex imitating the end of the U5 LTR, and various concentrations of compound 4 (in μM) as indicated at the top of the figure. No Int: absence of integrase; (D): DMSO; (21-mer): transferred strand; (19-mer): 3′ maturation product. The strand transfer products are indicated on the right.

FIG. 10: 3′ maturation and strand transfer activities for HIV-1 integrase in the presence of compound 4. IC50 value for graph; percentage inhibition of strand transfer (line) and inhibition of maturation (dotted line) were determined as a function of the concentration (M) of compound 4 (abscissa).

FIG. 11: Simplified example of a method for the synthesis of the molecules of the invention.

### EXAMPLES

A. Methods and Apparatus

Compounds

For this study, eight hundred compounds were supplied by the SPOT EA 3857 laboratory (laboratory for synthesis and organic and therapeutic physiochemistry) at François Rabelais University in Tours, France. The bis-(heteroaryl)maleimide derivatives (1-4 of FIG. 1) were synthesized by the laboratory of Professor Marie-Claude Viaud-Massuard as described in the literature (25). Twenty compounds were purchased from ChemBridge or from InterBioscreen, and have previously been described as Tn5 transposase inhibitors (14). Twenty-eight compounds have been described as HIV-1 integrase inhibitors; sixteen were DNA insertion compounds, graciously donated by Dr Vladimir Ryabinin (Institute of Molecular Biology, Koltsovo, Russia), five were novel thiazolothiazepins (26), four were G-quartets (27-30), and the final two were 3,5-dicafeoylquinic acid (31) and “integrase 3 inhibitor” (Merck). The active compounds are shown in FIG. 1. All of the compounds were taken up in suspension in DMSO.

Proteins

The full length transposase MOS1 [amino acids 1 to 345] and the MOS1 dimerisation domain [amino acids 1 to 85] were produced and purified in the form of a fusion protein bonded to maltose binding protein (MBP) as previously described (8). The terms “transposase” “MOS1” and “Tnp” used here indicate a single sub-unit of the protein Mos1.

In Vitro Transposition Tests

The in vitro transposition reactions were carried out using pBC-3T3 both as the donor DNA and as the target, as was carried out previously for transposition tests in bacteria (16). The transposition reaction mixtures contained 10 mM of Tris-HCl (pH 9), 50 mM of NaCl, 0.5 mM of dithiothreitol, 20 mM of MgCl₂, 0.5 mM of EDTA, 100 ng of BSA, 80 nM of MBP-MOS1 and 600 ng of supercoiled pBC-3T3 in a volume of 20 μl. Inhibitors (final concentration 80 μM) or DMSO were added to the reaction mixtures before the transposase. The reactions were carried out freely for 30 min at 30° C. then 10 μl of the stop solution (0.4% SDS, 0.4 μg/μl proteinase K) were added and the reaction mixtures were incubated at 37° C. for an additional 30 min, then at 65° C. for 10 min. The products were extracted with phenol-chloroform and precipitated in ethanol with 1 μg of yeast tRNA using conventional techniques. 10% of the reaction mixture was co-transformed with 0.01 ng of pBS-SK(−), used as a positive control for transformation (Stratagene), in 45 μl of electrocompetent Escherichia coli JM109 cells (2 mm cell, 1.5 kV in a MicroPulser™ from BioRad). The bacteria were cultivated at 37° C. for 1 hour in SOC medium. Appropriate dilutions of each reaction were spread on LB-ampicillin gelose (100 μg/ml) to evaluate the transformation efficacy and on LB-tetracyclin gelose (12.5 μg/ml) and LB-chloramphenicol gelose (80 μg/ml) in order to evaluate the frequency of transposition. The frequency of transposition is the number of Tet® colonies divided by the number of Chloram® colonies. The transformation efficacy was monitored from the number of Amp® colonies. The effect of the various compounds was expressed by calculating an inhibition factor (IF) such that IF=the frequency of transposition of the control (in DMSO) divided by the frequency of transposition obtained with the compound. Each experiment was repeated five times for the ten most effective drugs.

Electrophoretic Mobility Shift Tests (EMSA)

The ITR30 was prepared and labeled as described above (8) with certain minor modifications: after labelling, the DNA was purified further on a native polyacrylamide gel. The binding reactions were carried out in 50 mM of NaCl, 0.5 mM of DTT, 10 mM of Tris (pH 9), 5% of glycerol and 100 ng of BSA. Each 20 μl reaction contained 0.2 pmol of ITR30 labelled with ³²P, 400 mM of purified MBP-MOS1, 5 mM of EDTA, and 1 μl of the test compound (final concentration 400 μM) or DMSO. The mixtures were incubated at 30° C. for two hours. The complexes were separated using discontinuous native polyacrylamide gels with 4% to 6% of TBE 0.25× (acrylamide/bisacrylamide 30:0.93) containing 5% of glycerol. The gels were subjected to 200 V for 3 hours then autoradiographed.

In Vitro Excision Tests and Determination of CI50

These tests were carried out under the same conditions as for the in vitro transposition tests, with the exception of the fact that the incubation was carried out at 37° C. instead of 30° C. In order to determine the IC50, a range of concentrations of the compound from 1000 to 0.8 μM was used as indicated in the text. After incubation, the reaction was interrupted by adding 2 μl of 10× loading buffer and the products were loaded directly onto a 0.8% TAE 1×-agarose gel containing ethidium bromide. The quantity of backbone liberated was evaluated using the GeneSnap system (SynGene). The percentage inhibition of excision was plotted as a function of the concentration of the compound. The experimental data were adjusted on a sigmoid dose-response curve using Prism software. Each experiment was repeated at least two times.

Mos1 Strand Transfer Reactions

The reactions (40 μl) were carried out in 50 mM of Tris-HCl (pH 9), 0.1 mg/ml of BSA and 5% of glycerol in the presence of 10 nM of ITR substrate which had been cleaved and 80 nM of MBP-MOS1. The pre-cleaved ITR substrate had been formed by hybridizing the oligonucleotides NTS-3 and TS:

NTS-3:
5′-GGTGTACAAGTATGAAATGTCGTTTCG-3′;
and
TS:
5′-AATTCGAAACGACATTTCATACTTGTACACCTGA-3′.

TS was radiolabelled in the 5′ position with a polynucleotide kinase T4 (Promega) and α-[³²P]ATP. After 20 min at ambient temperature, MgCl₂ (final concentration 5 mM) and 200 ng of pBC-KS target plasmid (Stratagene) per reaction were added over 1 hour at 30° C. The inhibitors, diluted in 5% DMSO, were added during formation of the complexes and before adding the MgCl₂ and the target plasmid. The reactions were interrupted with 10 mM of EDTA and treated with 0.1 mg/ml of proteinase K, 0.1% of SDS and 2 mM of CaCl₂ at 45° C. for 1 hour. The strand transfer reactions were loaded and resolved on a 0.8% TBE 1× agarose gel. The gel was stained with ethidium bromide, then dried and exposed on a phosphorus screen (Phosphorimager, STORM Molecular Dynamics). The insertion products were quantified using Image Quant software.

3′ Maturation Tests and HIV-1 Strand Transfer Tests

HIV-1 integrase was purified as described in (32). 200 nM of HIV-1 integrase was incubated in the presence of 6.25 nM of radiolabelled oligonucleotide duplex imitating the end of the LTR U5 in a buffer containing 20 mM of HEPES (pH 7.5), 10 mM of NaCl, 4 mM of DTT, 7.5 mM of MgCl₂, 10% of DMSO and various concentrations of drugs. The reaction was carried out for 2 hours at 37° C. and the products were separated on an 18% acrylamide urea gel. The gel was scanned using a phosphorimager and analyzed using Image Quant (Molecular Dynamics) software. Each experiment was carried out in duplicate.

B. Results

Identification of MOS1 Inhibitors

During a first test for the identification of inhibitors of Mos1 transposase, a collection of 170 molecules was screened using an in vitro transposition test. The first part of the test panel was constituted by inhibitors of Tn5 transposase and of HIV-1 integrase. The other compounds, which had never been tested as integrase inhibitors or transposase inhibitors, were substituted heterocyclic derivatives.

The transposition test was carried out using a purified MOS1 protein fused with the MBP and the pBC-3T3 plasmid carrying a pseudo-Mos1 transposon. In this test, pBC-3T3 was used as the pseudo-Mos1 donor, or as the target for integration of the transposon (16). The transposition events were revealed by promoter labelling. The concentration of drugs used for the initial screening was 80 μM. The 10 best compounds had an inhibition factor (IF) of more than 25 (more than 96% inhibition) compared with the control experiment carried out in the absence of inhibitor, but in the presence of DMSO (Table II).

TABLE II
Frequency of transposition and inhibition factor for
the 10 best compounds (1 to 10 in FIG. 1); the values
for the transposition frequency and the inhibition factors
are the mean of at least five experiments.

| | Transposition | |
|---|---|---|
| Compounds | frequencies | Inhibition factors |
| DMSO | 4 × 10⁻⁴ | 1 |
| 1 | 1.09 × 10⁻⁶ | 367 |
| 2 | 1.2 × 10⁻⁶ | 33 |
| 3 | 4.01 × 10⁻⁶ | 100 |
| 4 | 5.37 × 10⁻⁶ | 75 |
| 5 | 8.38 × 10⁻⁶ | 48 |
| 6 | 1.01 × 10⁻⁵ | 40 |
| 7 | 4.85 × 10⁻⁶ | 82.5 |
| 8 | 5.04 × 10⁻⁶ | 79 |
| 9 | 1.64 × 10⁻⁵ | 25 |
| 10 | 2.76 × 10⁻⁶ | 145 |

The inhibition factors are the ratio between the transposition frequency obtained with DMSO and that obtained with each of compounds 1 to 10.

The structures of said inhibitors are shown in FIG. 1. Compounds 1 to 4 are bis-(heteroaryl)maleimide derivatives and constitute inhibitors for DDE/D enzymes unknown until now. Compounds 5 and 6 were N-methylpyrrole-polyamides, known to interact with DNA (synthesis in (17)). Compound 7 was a bis-coumarin derivative which have also been shown to be an inhibitor for Tn5 transposase and the HIV-1 integrase (14). Compounds 8, 9 and 10 were respectively a cinnamoyl derivative, a benzoic acid derivative and a thioxothiazolidine substituted with a carboxylic acid. The three compounds (8, 9 and 10) have already been identified as inhibitors of the Tn5 transposase (14).

Activity of Inhibitors on Excision of Transposon

For a better characterization of the inhibiting activity of these ten compounds and their action mechanisms on the transposition of Mos1, the capacity of the chemical compounds to inhibit excision of the transposon was tested in vitro, using the pBC-3T3 transposon as a donor plasmid. MOS1 triggers excision of the transposon from a donor plasmid, generating two linear DNA fragments, the transposon (1.2 kb) and the plasmid backbone (3.4 kb). After excision, the transposon can be re-inserted in a target. The excision activity of the drug was measured by quantifying the plasmid backbone as it is a final product in the reaction. The activity of compound 9 is shown in FIG. 3a. In the absence of a drug, the transposase released the transposon from the donor plasmid (line 8). At the highest concentration of the drug (1 mM), excision of the transposon was completely suppressed (line 2).

In order to determine the concentration of drug necessary to inhibit 50% of the reaction (IC50), the inhibition percentages measured from two independent experiments were plotted as a function of the concentration of drug (FIG. 3b). Similar experiments were carried out with the nine other chemical compounds and the corresponding IC50 values are shown in Table III.

TABLE III
Effect of each of the compounds on various steps for the Mos1
transposon; the IC50 values for the MOS1-ITR bond and for excision
of Mos1 are shown in μM, unless otherwise indicated.

| | | Assembly of | IC50 Exe. |
|---|---|---|---|
| Compounds | IC50 bond (μM) | complexes | (μM) |
| 1 | >400 | + | 18 < IC50 < 54 |
| 2 | 15 | − | 12 < IC50 < 15 |
| 3 | 30 | − | 7 < IC50 < 13 |
| 4 | 12 | − | 9 < IC50 < 18 |
| 5 | <50 nM | − | 5 < IC50 < 12 |
| 6 | <50 nM | − | 4 < IC50 < 22 |
| 7 | ND | − | 86 < IC50 < 91 |
| 8 | ND | − | 36 < IC50 < 44 |
| 9 | ND | +/− | 138 < IC50 < 166 |
| 10 | ND | − | 29 < IC50 < 39 |

The effects on the assembly of MOS1-ITR complexes are indicated by (+): complexes observed; (−): absence of complexes and (+/): traces of SEC1; ND: not determined.

The compounds 2 to 6 were the best inhibitors of excision, with IC50 values of the order of 10 μM. A second set of drugs, with IC50 values of 35 μM (compound 10) to 150 μM (compound 9) comprised the Tn5 inhibitors. Finally, bis-furyl-maleimide (compound 1), the best inhibitor characterized in the initial transposition test, inhibited excision with a IC50 in the range 18 to 54 μM. This IC50 value suggests that this compound could also act on the subsequent steps of the transposition.

These results indicate that compounds 1 to 10 have a substantial impact on transposon excision. Tn5 transposase inhibitors (compounds 7 to 10) block the formation of the synaptic complex of Tn5 (14), while compounds 5 and 6 are powerful inhibitors of DNA protein binding (synthesis in (17)). The data in the literature and these results strongly suggest that these compounds could act in the formation of transposase-ITR complexes, a step which occurs before excision.

Activity of Inhibitors in the Assembly of MOS-ITR Complexes

The capacity of the ten molecules to inhibit the appearance of complexes formed in vitro between MOS1 and ITR 3′ (8) was tested using the gel mobility shift technique (EMSA). MOS1-ITR complexes were formed with a radiolabelled ITR 3′ (ITR30) in the presence of a 400 μM concentration of molecule. This concentration was at least 2.5 times higher than the highest IC50 value measured for the ten compounds.

These initial data indicate that the complexes were still observed in the presence of compound 1 (FIG. 4, line 4) but that the motif was modified, which suggests that the drug interacts with the complexes. In contrast, the complexes were completely destabilized in the presence of compounds 2, 3, 4, 7, 8 and 10, and almost completely suppressed (90% inhibition) in the presence of compound 9, which had the highest IC50 value for the excision activity. Only traces of the abortive SEC1 complex were detected. The data observed here for compounds 7 to 10 are entirely in agreement with the mode of action of these molecules on the assembly of Tn5 transposase complexes and of HIV-1 integrase (14). Finally, a concentration of 400 μM of compounds 5 and 6 induced precipitation of the ITR substrates and the transposase complexes in the wells, probably by agglomeration or neutralization of the DNA load. A similar precipitation of the ITR substrate alone was also observed at high concentrations of drugs 5 and 6 in native PAGE (not shown).

Interactions between DNA and compounds 5 and 6 were also observed with the plasmid substrate during the excision test (FIG. 5). In order to confirm the mechanism of action of drugs 5 and 6, the experiments were repeated by reducing their concentration. These compounds completely blocked the formation of specific transposase-ITR complexes at low concentrations (40 nM, FIG. 6). In agreement with what is known of N-methylpyrrole-polyamides (17), these data thus indicate that compounds 5 and 6 bond with the ITR, thereby acting as competitors for MOS1.

Similar experiments were carried out using the novel transposase inhibitors (compounds 2 to 4) and these showed that these inhibitors block the formation of specific transposase-ITR complexes with a IC50 value similar to that for excision (Table III).

It was thus decided to confirm that the molecules which inhibit the formation of complexes do not inhibit the formation of MOS1 dimers, a step which is assumed to take place before binding to the ITR (18). By using a truncated form of MOS1 (Tnp[1-85]), the dimerisation was measured using glutaraldehyde binding experiments as described previously (18). None of the test compounds could inhibit the formation of Tnp[1-85] dimers in a concentration of 500 μM (not shown), which excludes the hypothesis whereby said compounds can inhibit the formation of complexes by destabilizing the N-terminal dimerisation domain. These results suggest that these inhibitors most probably act at the ITR-MOS1 interaction level. The data for compounds 5 to 10 are in agreement with the mechanism of the action of these compounds on Tn5 transposase and HIV-1 integrase, and so the study was focused onto the novel compounds 1 to 4.

Effect of Compounds 1 to 4 of FIG. 1 on the Strand Transfer Reactions

We have already shown that three of the novel compounds (compounds 2 to 4) impede the assembly of MOS-ITR complexes, while compound 1 acts at the Mos1 excision level. Excision and strand transfer reactions are closely related.

Compound 1 was thus tested to verify whether it could also inhibit the strand transfer reaction. Pre-cleaved ITRs were labelled at the 5′ end of the transfer strand and used to form strand transfer complexes. The inhibitor was added prior to the formation of MOS1-ITR complexes. Transposition events in a target plasmid were detected after resolving the reaction products on an agarose gel (FIG. 7). The percentage inhibition was determined for four independent experiments and plotted as a function of the concentration of drug (FIG. 8). Compound 1 inhibited the strand transfer reaction with a IC50 value in the range 19 to 45 μM. It also had the properties of a catalytic inhibitor; it could not prevent the assembly of MOS1-LTR complexes at concentrations that inhibited both the excision reaction and the strand transfer reaction.

Effect of Inhibitors on 3′ Maturation Activities and Strand Transfer of HIV-1 Integrase

The above results show that inhibitors of Tn5 and HIV-1 integrase can also inhibit MOS1. To verify whether the novel group of inhibitors (compounds 1 to 4) is also susceptible of blocking HIV-1 integrase, the activity of compound 4 was tested against the 3′ maturation and strand transfer activities of HIV-1 integrase. The test was carried out as described previously (19); the results are shown in FIG. 9. The percentage inhibition measured in the two independent experiments were traced as a function of the concentration of compound 4 (FIG. 10). Compound 4 inhibited 3′ maturation with a IC50 of 70 μM, and strand transfer activity with a IC50 of 7 μM; in other words this compound was 10 times more active against strand transfer activity. It was also an excellent inhibitor of HIV-1 integrase. This demonstrates that bis-(furanyl)-N-maleimide derivatives constitute a novel group of inhibitors with increased activity against the transposase of Mos1 and the HIV-1 integrase. In consequence, MOS1 can be considered as a novel substitute for the identification of inhibitors of HIV-1 integrase.

### Discussion

Identification of MOS1 Inhibitors

The availability of specific inhibitors is very important for the analysis and control of elaborate mechanisms such as transposition. For the first time, the present application characterizes chemical compounds which inhibit the transposition of mariner, the most widespread transposable element in eukaryotes. The mechanism of action of the most effective inhibitors, identified in detail by screening a panel of 170 compounds, has been studied. A first group was constituted by compounds which had already been identified as inhibitors of transposase (Tn5) or integrase (HIV-1 (compounds 5 to 10 in FIG. 1). All of these inhibitors block the formation of MOS1-ITR complexes. The second group is composed of a novel family of molecules with excellent inhibiting activities both against the transposase MOS1 and the HIV-1 integrase (compounds 1 to 4 in FIG. 1).

The target for the compounds means that a distinction can be drawn between compounds 5 and 6, which bind to DNA, and the other compounds which are deprived of the capacity to bind to DNA. Compounds 7 to 10 had a similar mechanism of action on MOS1 and Tn5 transposase. They inhibit the formation of complexes, probably by perturbing one of the DNA's transposase recognition domains. The Tn5 inhibitors were less effective against the transposition of Mos1, which may reflect the differences between the DNA recognition mechanisms and the organization of the complexes. Compounds 5 and 6 are hairpin polyamides composed of a N -methylpyrrole-polyamide bonded by a γ-aminobutyric acid (γ turn) and bonded to the minor DNA groove (17). They are specific of a short sequence constituted by three (compound 6) or four (compound 5) A/T pairs. These sequences are present at the end but also inside the ITR substrate. A single N-methylpyrrole with no γ-turn linker was also active against the transposition of MOS1 (IF˜20). They were also excellent inhibitors of the formation of complexes (IC50 of the bond<40 nM), but they were less effective in the presence of non-specific DNA. In the excision test, the quantity of DNA was 160 times higher than in the gel shift test, and the activity of the compounds fell by a factor of 250 (IC50 of excision=10 μM).

Bis-(heteroaryl)maleimide Derivatives Constitute a Novel Backbone for DDE Enzyme Inhibitors

The panel of test compounds in the context of this application as MOS1 inhibitors was principally constituted by heterocyclic molecules substituted with hydroxyl groups. Small molecules of this type had previously been identified as effective inhibitors of HIV-1 integrase (20). Of the set of molecules tested in the context of this application, 21 were derivatives of bis-(heteroaryl)maleimide. The most effective inhibitors which came to light were all organized about a bis-(heteroaryl)maleimide backbone (compounds 1 to 4 of FIG. 1). It has been shown that the unsubstituted backbones were ineffective, which suggests that the bis-(heteroaryl)maleimide backbone itself is not the pharmacophore (data not shown). N-substitution of the maleimide group and/or substitution of the heteroaryl group both had a major impact on the mechanism of action and the efficacy of the compounds. Compounds 2, 3 and 4, which are all N-substituted, inhibited assembly of MOS1-ITR complexes with IC50 values very similar to those of excision. In contrast, compound 1, which was not N-substituted, did not inhibit assembly of the MOS1-ITR complexes even though the differences in the migration motif suggest that compound 1 might modify the structure of the complexes. This compound was active both against excision and strand transfer activities, and exhibited IC50 values which were slightly higher than those of compounds 2, 3 and 4. The compounds of this family do not bind to DNA (21), but they probably target transposase or transposase-DNA complexes. There are two possible explanations for the radical difference in inhibition mechanism between compound 1 and the other maleimide derivatives. Firstly, the target for the drug may be different, due to substitution by an aromatic group of the N-maleimide (compounds 2 to 4), which could explain the difference in inhibition mechanism. Secondly, the four compounds could all have a similar target, but the voluminous aromatic substitution of the N-maleimide perturbs the DNA bond, while the non-N-substituted compound tolerates the presence of DNA.

As a target, the DDD catalytic pocket of MOS1 transposase constitutes an excellent candidate for explaining the major change in the inhibition mechanism and cross-activity of these compounds against HIV-1 integrase, as it includes similar catalytic residues and binds to the intersection of two DNA substrates involved in the reaction. Compounds provided with a backbone that can bind to this region could have different inhibitor phenotypes, becoming specific inhibitors of complex formation, excision or strand transfer as a consequence of minor substitutional differences in their backbone.

The various targets for the compounds could also explain the various phenotypes of this family of compounds. Point mutations in the N-terminal domain of HIMAR1 (a mariner transposase related to MOS1) illustrate the manner in which changes in the transposase-ITR interactions could have pleiotropic consequences. Certain mutations completely destabilize the transposase-ITR complexes, while others allow complexes to form but inhibit the first or the second strand cleavage reaction (22). This implies that the compounds that target the N-terminal domain/ITR interaction could reflect the phenotype of the mutations in the N-terminal domain. Identification of the target(s) of this family of compounds is already under way, as it appears to be essential to future developments and to understanding the mechanism of action of these inhibitors on the transposition of Mos1 and the HIV-1 integration process.

Cross-Activity of DDE Enzyme Inhibitors

The capacity of a compound to inhibit enzymes related as regards mechanisms (cross activity) is illustrated by the compounds which inhibit HIV-1 integrase, ASV integrase and other related retroviral integrases (23). Further, integrase inhibitors of the diketoacid type (5 CITEP and L-708 906) are also active against distantly related RAG recombinases (24), although the activity of this inhibitor is greatly reduced (by a factor of 100). More recently, Tn5 transposase inhibitor screening was used to identify compounds active against HIV-1 integrase (14).

The studies presented in the present application constitute a novel demonstration of the cross activity between the inhibitors of Tn5 and MOS1 transposases and for the first time between MOS1 and HIV-1 integrase. The cross activity of the novel family of identified inhibitors shows that MOS1 constitutes an excellent substitute for inhibitor screening. This substitute is of great interest for the following reasons. Firstly, the transposition of MOS1 functions in an eukaryotic environment, like integrase, which opens up the possibility for an inhibition test in eukaryotic cells. Secondly, the structure of the catalytic domain of MOS1 is very similar to that of retroviral integrases of ASV and HIV-1 (9, 10). Thirdly, the catalytic process of DNA cleavage in transposition is similar to the reaction of maturation of a retroviral integrase, the reaction of cleavage of a second strand imitating the 3′ maturation activity of integrase (there is no inter-strand intermediate or hairpin in these two reactions), Until now, the Tn5 transpososome has been used as a model for retroviral integrasome, but the mariner transposome could constitute a novel, probably better, model for the organization of HIV-1 integrasome. Nevertheless, it must be remembered that these two models (Mos1 and HIV-1) exhibit substantial differences, such as the structure and configuration of their DNA binding domain, the nature of the donor DNA substrate and the DNA cleavage reaction (cleavage of two strands as opposed to the cleavage of a single strand), which means that the two systems are not entirely interchangeable. In this regard, the use of drugs with cross activity will offer an important tool in detecting similarities and differences between these two enzymes.

### BIBLIOGRAPHY

1. Craig N L C R, Gellert M, Lambowitz A M (2002) Mobile DNA II (ASM Press, Washington, D.C.).
2. Duverger Y, Belougne J, Scaglione S, Brandli D, Beclin C, & Ewbank J J (2007) Nucleic Acids Res 35, e11.
3. Keravala A, Liu D, Lechman E R, Wolfe D, Nash J A, Lampe D J, & Robbins P D (2006) Hum Gene Ther 17, 1006-1018.
4. Kapetanaki M G, Loukeris T G, Livadaras I, & Savakis C (2002) Nucleic Acids Res 30, 3333-3340.
5. Kuwayama H, Yaginuma T, Yamashita O, & Niimi T (2006) Insect Mol Biol 15, 507-512.
6. Yant S R, Huang Y, Akache B, & Kay M A (2007) Nucleic Acids Res.
7. Koga A & Hori H (2001) Genes Genet Syst 76, 1-8.
8. Auge-Gouillou C, Brillet B, Hamelin M H, & Bigot Y (2005) Mol Cell Biol 25, 2861-2870.
9. Richardson J M, Finnegan D J, & Walkinshaw M D (2007) Acta Crystallograph Sect F Struct Biol Cryst Commun 63, 434-437.
10. Richardson J M, Dawson A, O'Hagan N, Taylor P, Finnegan D J, & Walkinshaw M D (2006) Embo J 25, 1324-1334.
11. Curcio M J & Derbyshire K M (2003) Nat Rev Mol Cell Biol 4, 865-877.
12. Rice P A & Baker T A (2001) Nat Struct Biol 8, 302-307.
13. Dyda F, Hickman A B, Jenkins T M, Engelman A, Craigie R, & Davies D R (1994) Science 266, 1981-1986.
14. Ason B, Knauss D J, Balke A M, Merkel G, Skalka A M, & Reznikoff W S (2005) Antimicrob Agents Chemother 49, 2035-2043.
15. Barreca M L, De Luca L, Iraci N, & Chimirri A (2006) J Med Chem 49, 3994-3997.
16. Auge-Gouillou C, Hamelin M H, Demattei M V, Periquet M, & Bigot Y (2001) Mol Genet Genomics 265, 51-57.
17. Dervan P B E B (2003) Curr Opin Struct Biol 13, 284-299.
18. Auge-Gouillou C, Brillet B, Germon S, Hamelin M H, & Bigot Y (2005) J Mol Biol 351, 117-130.
19. Craigie R, Mizuuchi K, Bushman FD, & Engelman A (1991) Nucleic Acids Res 19, 2729-2734.
20. Dayam R & Neamati N (2003) Curr Pharm Des 9, 1789-1802.
21. Dubernet M (2004) in UFR sciences pharmaceutiques (University François Rabelais, Tours).
22. Butler M G, Chakraborty S A, & Lampe D J (2006) Genetica 127, 351-366.
23. Lubkowski J, Yang F, Alexandratos J, Wlodawer A, Zhao H, Burke T R, Jr., Neamati N, Pommier Y, Merkel G, & Skalka A M (1998) Proc Natl Acad Sci USA 95, 4831-4836.
24. Paull T T & Gellert M (2000) Proc Natl Acad Sci USA 97, 6409-6414.
25. Dubernet M C V, Guillard J and Viaud-Massuard M C (2005) tetrahedron 61, 4585-4593.
26. Aiello F, Brizzi A, Garofalo A, Grande F, Ragno G, Dayam R, & Neamati N (2004) Bioorg Med Chem 12, 4459-4466.
27. Jing N (2000) Expert Opin Investig Drugs 9, 1777-1785.
28. Jing N, De Clercq E, Rando R F, Pallansch L, Lackman-Smith C, Lee S, & Hogan M E (2000) J Biol Chem 275, 3421-3430.
29. Jing N, Marchand C, Liu J, Mitra R, Hogan M E, & Pommier Y (2000) J Biol Chem 275, 21460-21467.
30. de Soultrait V R, Lozach P Y, Altmeyer R, Tarrago-Litvak L, Litvak S, & Andreola M L (2002) J Mol Biol 324, 195-203.
31. Islam M S Y M, Yahara S, Okuno S, Ishiguro K, Yamakawa O (2002). J Agric Food Chem 50, 3718-3722.
32. Leh H, Brodin P, Bischerour J, Deprez E, Tauc P, Brochon J C, LeCam E, Coulaud D, Auclair C, & Mouscadet J F (2000) Biochemistry 39, 9285-9294.

1. A compound comprising a bis(heteroaryl)maleimide structure of formula (I): 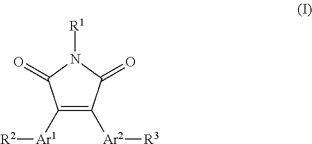
in which:
R¹ is selected from the group consisting of a hydrogen, a linear or branched alkyl, an alkenyl, an alkynyl, a cycloalkyl, a heterocyclyl, a heteroaryl, a heteroaralkyl, a heteroalkoxy, a carboxyl, an alkoxycarbonyl, a tetrazolyl, an acyl, an arylsulphonyl, a heteroarylsulphonyl, a phenyl, a hydroxyphenyl and a 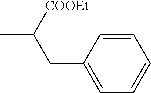
group;
R² and R³, independently of each other, are selected from the group consisting of a hydrogen, a carboxylic acid, a cyano, an oxime, an oxime ether, a tetrazole, an ester, a substituted or unsubstituted amide, an acid, a dibasic acid, a cycloalkylcarboxylic acid, a substituted or unsubstituted aryl, a substituted or unsubstituted heteroaryl, and a —C═CH—CHO group;
Ar¹ and Ar², independently of each other, are selected from the group consisting of a substituted or unsubstituted cycloalkyl, a substituted or unsubstituted heteroaryl, a substituted or unsubstituted aryl, and a linear or branched heteroalkyl;
with the proviso that R¹, R² and R³ must not all simultaneously be H.

2. The compound according to claim 1, in which R² and R³ are identical.

3. The compound according to claim 1, in which Ar¹ and Ar² are identical.

4. The compound according to claim 1, in which R² and R³ are identical, and Ar¹ and Ar² are identical.

5. The compound according to claim 1, in which:
Ar¹ is a monocyclic heteroaryl carrying R², selected from the group consisting of R²-pyridyl, R²-pyrazinyl, R²-furanyl, R²-thienyl, R²-pyrimidinyl, R²-isoxazolyl, R²-isothiazolyl, R²-oxazolyl, R²-thiazolyl, R²-pyrazolyl, R²-furazanyl, R²-pyrrolyl, R²-pyrazolyl, R²-triazolyl, R²-pyrazinyl and R²-pyridazinyl, said monocyclic heteroaryl being substituted or unsubstituted; and
Ar², independently of Ar¹, is a monocyclic heteroaryl carrying R³, selected from the group consisting of R³-pyridyl, R³-pyrazinyl, R³-furanyl, R³-thienyl, R³-pyrimidinyl, R³-isoxazolyl, R³-isothiazolyl, R³-oxazolyl, R³-thiazolyl, R³-pyrazolyl, R³-furazanyl, R³-pyrrolyl, R³-pyrazolyl, R³-triazolyl, R³-pyrazinyl and R³-pyridazinyl, said heteroaryl monocyclic being substituted or unsubstituted.

6. The compound according to claim 1, in which either Ar¹ or Ar², or each of Ar¹ and Ar² is, a furan group.

7. The compound according to claim 5 of formula (II), in which each of Ar¹ and Ar² is a furan group:

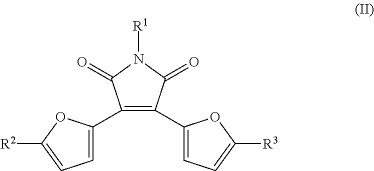

8. The compound according to claim 5, in which Ar¹ and Ar² are respectively R²-furanyl and R³-furanyl.

9. The compound according to claim 7, in which R² and R³ are identical.

10. The compound according to claim 1, in which either R² or R³ or each of R² and R³ is COOH.

11. The compound according to claim 1, in which either R² or R³ or each of R² and R³ is a C═CH—CHO group.

12. The compound according to claim 1, in which R¹ is a phenyl group, which may be unsubstituted or substituted.

13. The compound according to claim 12, in which R¹ is 4-hydroxyphenyl.

14. The compound according to claim 1, in which R¹ is a 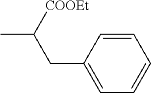
group.

15. The compound according to claim 1 in which R¹ is H.

16. The compound according to claim 1, with the further proviso that:
when R¹ is a hydrogen or a phenyl and Ar¹ and Ar² are furans, R² and R³ are each CHO; and

17. The compound according to claim 1 characterized in that it is N-substituted.

18. The compound according to claim 15, characterized in that it is not N-substituted.

19. The compound according to claim 1, selected from the group consisting of the compounds of formulae (III) to (VI) below:

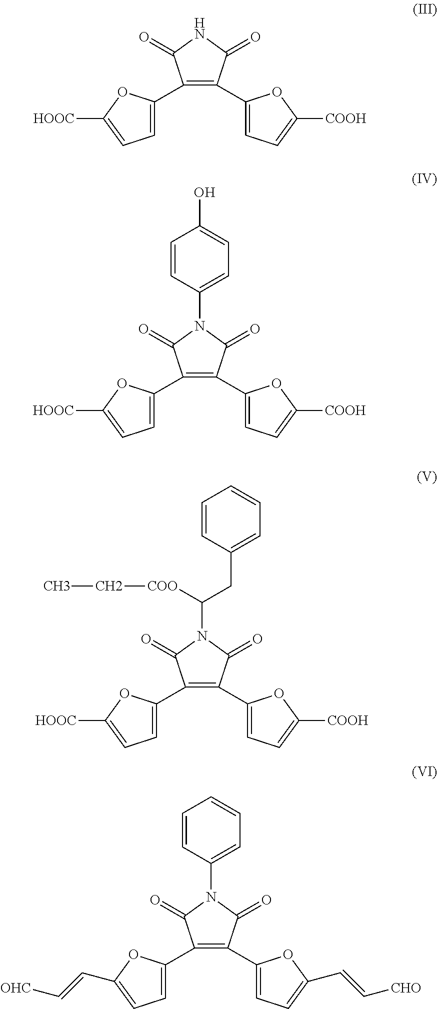

20. The compound according to claim 1 having a DDE/DDD enzyme inhibiting activity in vitro.

21. A composition, that comprises at least one compound according to any claim 1.

22. The composition according to claim 21, that further comprises a pharmaceutically acceptable vehicle.

23. The composition according to claim 21 that further comprises an additional compound which is biologically active in the treatment of symptoms linked to acquired immunodeficiency syndrome.

24. A method of in vitro inhibiting the activity of a DDE/DDD enzyme which comprises contacting the enzyme under suitable conditions with the compound according to claim 1.

25. (canceled)

26. (canceled)

27. A method of ex vivo inhibiting a transposase which comprises contacting the transposase under suitable conditions with the compound according to claim 1.

28. (canceled)

29. A method of in vivo inhibiting retroviral integrases which comprises contacting the retroviral integrases under suitable conditions with the compound according to claim 1.

30. (canceled)

31. A method of reducing or suppressing replication of a retrovirus which comprises contacting the retrovirus under suitable conditions with the compound according to claim 1.

32. (canceled)

33. A method according to claim 31, wherein said retrovirus is the HIV virus (Human Immunodeficiency Virus).

34. (canceled)

35. (canceled)

36. A method of treating a subject suffering from symptoms associated with an infection by a HIV which comprises administering to the subject the compound according to claim 1.

37. (canceled)

38. (canceled)

39. (canceled)

40. (canceled)

41. (canceled)

42. (canceled)

43. The composition according to claim 21 in the form of a solid (cachet, powder, gelule, pill, suppository, quick release tablet, gastro-resistant tablet, delayed release tablet) or liquid (syrup, injectable solution, eye wash).

44. The method according to claim 36, wherein the administration is effected orally, buccal-transmucosally, vaginally, rectally, parenterally (intravenously, intramuscularly or subcutaneously), transcutaneously (transdermally or percutaneously) or cutaneously.

45. (canceled)

46. Use of the MOS1 system for screening retroviral integrase inhibitors.

47. (canceled)

48. (canceled)
